(19) United States
(12) Patent Application Publication
(10) Pub. No.: US 2011/0112375 A1
(43) Pub. Date: May 12, 2011

(54) PORTAL APPARATUS INCLUDING CONFORMABLE CUP SEAL

(75) Inventor: Michael J. Bettuchi, Middletown, CT (US)

(73) Assignee: Tyco Healthcare Group LP

(21) Appl. No.: 12/908,958

(22) Filed: Oct. 21, 2010

Related U.S. Application Data
(60) Provisional application No. 61/260,485, filed on Nov. 12, 2009.

(51) Int. Cl. A61B 1/32

(52) U.S. Cl. 600/210

(57) ABSTRACT

A surgical portal system for a surgical procedure incorporating the use of one or more sutures includes a portal housing and a portal sleeve extending from the portal housing. The portal housing includes an internal wall. The portal housing and the portal sleeve define a longitudinal axis and has a longitudinal opening for passage of a surgical instrument and one or more sutures. The portal sleeve is dimensioned for insertion within tissue to provide access to an underlying body site. A seal is mounted to the internal wall of the portal housing and arranged to intersect the longitudinal axis. The seal comprises a conformable material dimensioned and configured to substantially conform about the surgical instrument upon passage of the surgical instrument through the longitudinal opening, to establish a substantial seal thereabout and to displace in at least a radial direction to contact the internal wall of the portal housing to engage one or more sutures extending through the longitudinal opening and to establish a substantial sealing relation with the internal housing wall in the presence of the one or more sutures.

### CROSS-REFERENCE TO RELATED APPLICATIONS

This application claims priority to and the benefit of, U.S. Provisional Application Ser. No. 61/260,485, filed on Nov. 12, 2009, the entire contents of which are incorporated herein by reference.

### BACKGROUND

1. Technical Field

The present disclosure relates to a portal apparatus, and, more particularly, relates to a portal apparatus incorporating a conformable cup-shaped seal adapted to secure one or more sutures in a predefined orientation while also potentially maintaining a substantial sealed relation with the interior of the portal apparatus.

2. Description of the Related Art

Trocars and other surgical portal apparatii are known, as are a myriad of procedures that may be preformed using such apparatii. Many of the minimally invasive procedures performed through portals necessitate or are simplified by the use of one or more sutures passing through the surgical portal apparatus. Sutures extending into a body cavity through a surgical portal may be used to, for example, temporarily retain tissue, manipulate tissue, anchor tissue or operate peripheral devices.

In an attempt to reduce the number of incision sites required to complete a given surgical procedure, a single surgical portal apparatus may be used to pass one or more sutures into a body cavity, in addition to providing access for one or more devices. A single anchor device (e.g., bone anchor) may have numerous suture ends extending from the surgical site and through the portal passage. The sutures, however, are subject to potential entanglement or confusion of receptive suture ends. Another concerns the ability to establish or maintain a seal within the portal in the presence of the suture and/or surgical instrument to prevent insufflation fluids or irrigant fluids from escaping through the portal.

### SUMMARY

Accordingly, a surgical portal system for a surgical procedure incorporating the use of one or more sutures includes a portal housing and a portal sleeve extending from the portal housing. The portal housing includes an internal wall. The portal housing and the portal sleeve define a longitudinal axis and a longitudinal opening for passage of a surgical instrument and one or more sutures. The portal sleeve is dimensioned for insertion within tissue to provide access to an underlying body site. The portal seal is mounted to the internal housing wall of the portal housing. The portal seal includes a proximal seal wall arranged to intersect the longitudinal axis and a peripheral seal wall depending from the proximal seal wall in an at least axial direction. The portal seal is adapted to deflect during passage of the surgical instrument through the longitudinal opening whereby wall segments of the peripheral seal wall are displaced in at least a radial direction to contact the internal housing wall of the portal housing to engage one or more sutures extending through the longitudinal opening. The peripheral seal wall of the portal seal may be dimensioned whereby during passage of the surgical instrument the wall segments of the peripheral seal wall conform to secure the one or more sutures in general fixed relation against the internal housing wall. Alternatively, or additionally, the peripheral seal wall may be dimensioned whereby during passage of the surgical instrument the wall segments of the peripheral seal wall establish a substantial sealing relation with the internal housing wall against the one or more sutures.

The peripheral seal wall may be obliquely arranged with respect to the longitudinal axis when in an initial condition thereof. The portal seal may be in the general shape of a frustum. As a further embodiment, the portal seal defines an open cavity or a cavity which is open adjacent to or leading toward the portal sleeve.

The proximal seal wall of the portal seal may be devoid of a seal passage. The portal seal may be mounted to the internal housing wall with a hinge, and be adapted for pivotal movement about the hinge. The portal seal may comprise a conformable material adapted to conform about the surgical instrument to establish a substantial sealed relation therewith. The portal seal may comprise a conformable material adapted to conform about the one or more sutures to establish a substantial sealed relation therewith and the internal housing wall.

The portal system may include an instrument seal. The instrument seal has inner surfaces adapted to establish a substantial sealed relation with the surgical instrument. The portal system further may include a zero closure valve adapted to substantially close the longitudinal opening in the absence of the surgical instrument.

A method for performing a surgical procedure is also disclosed.

### BRIEF DESCRIPTION OF THE DRAWINGS

Various embodiments of the present disclosure are described herein with reference to the drawings wherein:

FIG. 1 is a side cross-sectional view of an portal apparatus including a housing with an portal seal according to an embodiment of the present disclosure;

FIG. 2 is a partial cross-sectional view of a proximal end of the portal apparatus and the portal seal of FIG. 1; and

FIGS. 3A-3C is respective side views of the housing and the portal seal of FIGS. 1 and 2 shown prior to, during and after insertion of the surgical instrument.

### DETAILED DESCRIPTION

The apparatus and system herein disclosed may be configured for use in various surgical procedures, including laparoscopic, endoscopic, arthroscopic and orthopedic surgery. The apparatus provides passage between a subject's body cavity and the outside atmosphere and is capable of receiving surgical instruments of various sizes and configurations. Embodiments of the presently disclosed suture management systems are configured to receive, for example, clip appliers, graspers, dissectors, retractors, staplers, laser probes, photographic devices, suture anchor installation devices, endoscopes and laparoscopes, tubes, and the like. Such instruments are collectively referred to herein as “instruments” or “instrumentation.”

In addition to the instruments, the apparatus incorporates a seal which may conform about a surgical object while maintaining a seal within the apparatus interior even in the presence of sutures during, e.g., in an endoscopic procedure, laparoscopic procedure or arthroscopic procedure. The seal may also secure the sutures in a fixed position relative to the apparatus, which may facilitate organization of the suture ends. The seal may also be used to maintain the one or more sutures in a taut condition even in the presence of insufflation gases and/or saline fluids introduced within the operative site during, e.g., an arthroscopic procedure.

Referring now to the drawings wherein like reference numerals illustrate similar components throughout the several views, there is illustrated a surgical portal apparatus in accordance with the principles of the present disclosure. In the following description, the term “proximal” refers to the portion of the portal apparatus that is closest to the clinician, whereas the term “distal” refers to the portion of the portal apparatus that is farthest from the clinician. As used herein, the term “subject” refers to a human patient or other animal. The term “clinician” refers to a physician, nurse or other care provider and may include support personnel.

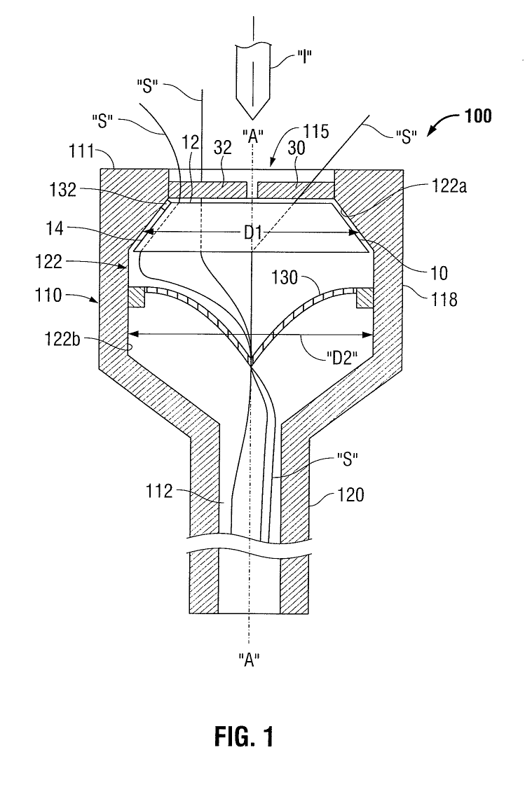

Referring to FIGS. 1-3, and initially with reference to FIG. 1, a portal seal 10 configured for use with a portal apparatus designated 100 is shown. Apparatus 100 and operative components associated therewith may be formed from any suitable material, e.g., a biocompatible material. Portal apparatus 100 defines a longitudinal axis “A” and includes a housing 110 and an access or portal member 120. A proximal end 111 of housing 110 includes an opening 115 and portal member 120 defines a longitudinal passageway 112. Opening 115 and longitudinal passageway 112 are generally aligned with respect to the longitudinal axis “A” to permit passage of surgical objects such as instruments “I” and/or sutures “S” utilized in connection with the procedure. Housing 110 includes outer and inner walls 118 and 122, respectively. Inner wall 122 includes a tapered segment, e.g., a generally frustoconical segment 122a having a first average or median diameter “D₁” and a cylindrical distal segment 122b having second diameter “D₂”, wherein the first diameter “D₁” is less than second diameter “D₂”. This frustoconical configuration when combined with portal seal 10 may provide a substantially fluid tight seal about the proximal end 111 in the presence or absence of a surgical instrument within portal apparatus. Additionally, the relatively long sloped surfaces associated with the frustoconical configuration may assist to reduce the escape of insufflation gases when one or more type of sutures “S” is present between the inner wall 122 of the housing 110 and the portal seal 10, such as, for example, during an arthroscopic surgery.

Portal seal 10 may be configured to provide a substantial seal between a body cavity of a patient and the outside atmosphere both during and subsequent to insertion of a surgical instrument “I” and or sutures “S” through the portal member 120. Portal seal 10 may accommodate instruments of varying diameter while providing a substantially fluid tight seal within the portal apparatus 100. Portal seal 10 may minimize egress of insufflation gases or irrigant fluids (e.g., in an arthroscopic procedure) through portal apparatus 10 during the procedure. It is also envisioned that a second seal such as a zero-closure seal 130 or duck bill seal can be used in conjunction with portal seal 10. The zero closure seal 130 may close in the absence of an object. The versatility of the presently disclosed portal seal 10 facilitates endoscopic surgery, wherein a variety of instruments having different diameters are often needed during a single surgical procedure.

The portal seal 10 may comprise a conformable material and may be configured to establish a seal within portal housing 110 during insertion and manipulation of a surgical object thereof. Suitable conformable materials include elastomeric synthetic and natural materials such as polyisoprene, natural rubber, butyl rubber, copolymers of isobutylene and isoprene, polybutadiene, polyurethane. The portal seal may include the fabric and elastomeric seal disclosed in commonly assigned U.S. Pat. No. 6,702,787 to Racenet, the entire contents of such disclosure being hereby incorporated by reference herein.

Portal seal 10 may conform to the internal surfaces of the wall 122 of the portal housing 110. More particularly, the portal seal 10 may be formed from any material that allows the portal seal 10 to transition between an initial condition in the absence of the surgical instrument “I” to an activated condition, wherein segments of the portal seal 10 are configured to deflect or deform when the surgical instrument “I” is inserted through the housing 110 and engage one or more sutures “S” extending through the portal apparatus 10 to secure the sutures in fixed relation, and, potentially, establish an internal seal within the portal housing and/or about the surgical instrument “I”.

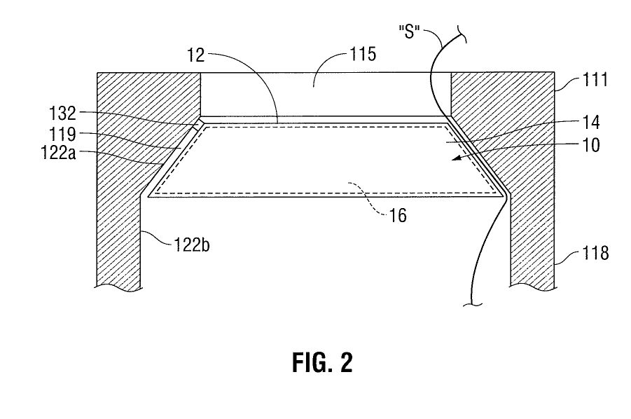

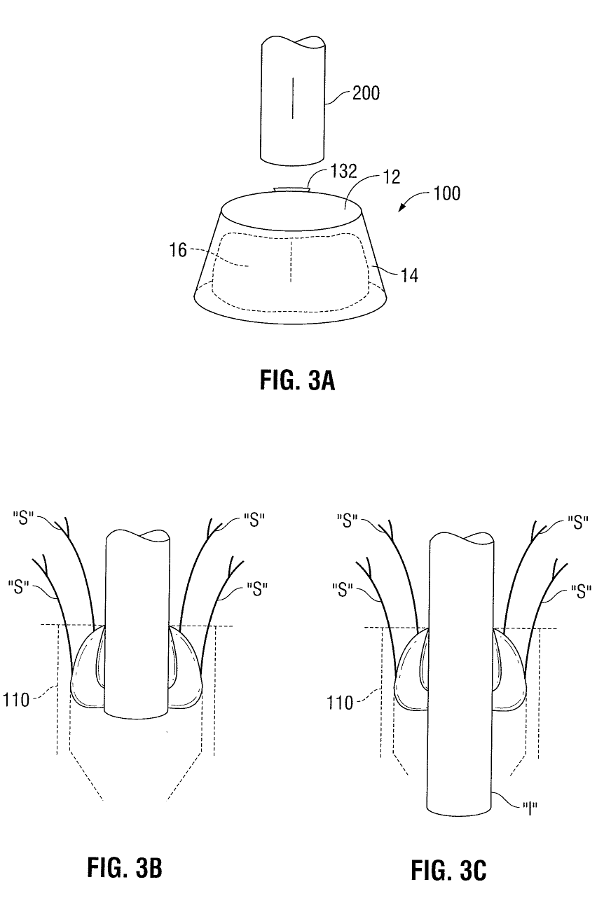

Referring now to FIGS. 3A-3C, in conjunction with FIGS. 1-2, portal seal 10 includes proximal seal wall 12 arranged to intersect the longitudinal axis “A-A” and outer peripheral wall 14 depending from the proximal seal wall 12 in an axial direction. In embodiments, proximal seal wall 12 is substantially planar and is arranged in orthogonal relation with the longitudinal axis “A-A”. Proximal seal wall 12 may be substantially solid, i.e., devoid of an aperture. Outer peripheral wall 14 is obliquely arranged with respect to the longitudinal axis “A-A”. In one embodiment, outer peripheral wall 14 and tapered segment 122a of inner wall 122 defines a substantially equivalent angle relative to the longitudinal axis “A-A”. In one embodiment, portal seal 10 defines a general hollow cup-shape or may be in the shape of a frustum. Outer peripheral wall 14 defines an open cavity 16 within the confines of the wall 14 which opens in the direction of portal sleeve 120.

In one embodiment, a slight annular space 119 may be provided between portal seal 10 and inner wall 122 of housing 110 in the absence of the surgical instrument. The annular space 119 may permit passage of the sutures “S” utilized during the procedure. In another embodiment, portal seal 10 engages the inner wall 122 including frusto-conical segment 122a and/or cylindrical segment 122b in substantial sealed relation in the absence of the surgical instrument “I”. In this embodiment, the frustoconical configuration when combined with the frustoconical configuration of inner wall 122 of the housing 110 may provide a substantially fluid tight seal about the proximal end 111 when a surgical instrument “I” is not inserted within the housing 110. Additionally, the relatively long sloped surfaces associated with the frustoconical configuration helps to reduce the escape of insufflation gases or saline when one or more type of sutures “S” is present between the inner wall 122 of the housing and the portal seal 10, such as, for example, when the portal apparatus 100 is used during an arthroscopic surgery.

In embodiments, portal seal 10 is connected to housing 110 through hinge 132 Hinge 132 may be any suitable type of hinge. Hinge 132 is configured to prevent the portal seal 10 from detaching from the inner wall 122 of the housing 110 when the surgical instrument “I” is being inserted through and removed from the housing 110. Hinge 132 may also permit portal seal 10 to pivot or flex during insertion and/or manipulation of the surgical instrument.

In embodiments, the entire portal seal 10 or a portion thereof may be coated with a material (e.g., polytetrafluoroethylene (PTFE) commercially available under the trademark TEFLON®) that provides the portal seal 10 with a relatively slippery surface to reduce laparoscopic instrument insertion forces. Alternatively, or in combination therewith, portal seal 10 may be made from a material that is not as lubricious.

In use, portal member 120 with an obturator (not shown) positioned therein may be used to create an incision to gain access to an underlying body cavity. The underlying cavity may be filled with insufflation gases if used during a laparoscopic procedure. In an arthroscopic procedure, fluids may be used to expand a joint such as the knee, shoulder or the like. An anchor or screw may be passed through the portal member 120 and secured within hard tissue as is known in the art. One suitable anchor and installation tool is disclosed in commonly assigned U.S. Pat. Nos. 5,944,739 and 5,993,459, the entire contents of each disclosure being hereby incorporated by reference herein. The portal seal 10 may establish a seal about the installation tool as previously discussed. The installation tool may be removed leaving the anchor or screw with suture segments “S” extending therefrom. The suture ends or segments “S” may be passed about the portal seal 10 through, e.g., the annular space 119 and through the proximal end 111 of the housing 110. In one embodiment, outer peripheral wall 14 of portal seal 10 may engage the sutures “S” to retain the sutures “S” in fixed relation within portal apparatus 10. Another surgical instrument “I” may be inserted through opening 115 of housing 110. Due to the conformable nature of the portal seal 10 and the lack of any seal passage through proximal seal wall 12, the wall segments of peripheral wall 14 and proximal wall 12 of portal seal 10 will deflect or deform accompanied by some pivotal movement of seal 10 about hinge 132 to 1) conform and establish a substantially fluid tight seal about the periphery of the instrument “I” (FIGS. 3B and 3C) while also accommodating the outer diameter of the suture ends, and/or 2) secure the suture ends within the portal housing. Moreover, the introduction of the surgical instrument displaces seal portions of the portal seal 10 and forces the portal seal 10 (and suture ends “S”) against frusto conical segment 122a and/or cylindrical distal segment 122b of the inner wall 122. While instrument “I” is disposed within the housing 110, portal seal 10 may maintain a substantially fluid tight seal about the instrument “I” such that the fluids (e.g., insufflation gases or saline fluids) within the body cavity are substantially prevented from escaping through the access assembly. A surgical procedure or task may be performed with the instrument “I”. As noted above, the frustoconical configuration of the portal seal 10 and inner sidewall 122 of housing 110 reduces the escape of insufflation gas or irrigant fluid when the sutures “S” are present and positioned between the portal seal 10 and inner sidewall 122. The sutures “S” are also retained in a predetermined relation within inner wall 122 of housing 110, which may facilitate suture organization and identification and avoid entanglement.

As a further alternative, portal seal 10 may include instrument seal 30 having a passage 32 adapted to establish a seal about the surgical instrument. A suitable instrument seal 30 may be the seal disclosed in the Racenet '787 patent mentioned hereinabove. (FIG. 1). The instrument seal 30 may be mechanically coupled to housing 110 in a manner to permit passage of sutures “S” therethrough, e.g., about the periphery of the instrument seal 30.

From the foregoing and with reference to the various drawings, those skilled in the art will appreciate that certain modifications can also be made to the present disclosure without departing from the scope of the same. For example, while portal seal 10 has been described herein as being fixedly secured to housing 110, it is within the purview of the present disclosure to provide portal seal 10 detachably mountable to the proximal end 111 of housing 110, e.g., for cleaning or disposal. In addition, portal seal 10 may be readily adapted for mounting to conventional cannulas of differing structures. The detachability of portal seal 10 from housing 110 may facilitate specimen removal through portal member 120.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical portal system for a surgical procedure incorporating the use of one or more sutures, which comprises:
a portal housing and a portal sleeve extending from the portal housing, the portal housing including an internal housing wall, the portal housing and the portal sleeve defining a longitudinal axis and having a longitudinal opening for passage of a surgical instrument and one or more sutures, the portal sleeve dimensioned for insertion within tissue to provide access to an underlying body site; and
a portal seal mounted to the internal housing wall of the portal housing, the portal seal including a proximal seal wall arranged to intersect the longitudinal axis and a peripheral seal wall depending from the proximal seal wall in an at least axial direction, the portal seal adapted to deflect during passage of the surgical instrument through the longitudinal opening whereby wall segments of the peripheral seal wall are displaced in at least a radial direction to contact the internal housing wall of the portal housing to engage one or more sutures extending through the longitudinal opening.

2. The portal system according to claim 1 wherein the peripheral seal wall of the portal seal is dimensioned whereby during passage of the surgical instrument the wall segments of the peripheral seal wall conform to secure the one or more sutures in general fixed relation against the internal housing wall.

3. The portal system according to claim 1 wherein the peripheral seal wall of the portal seal is dimensioned whereby during passage of the surgical instrument the wall segments of the peripheral seal wall establish a substantial sealing relation with the internal housing wall against the one or more sutures.

4. The portal system according to claim 1 wherein the peripheral seal wall seal of the portal seal is obliquely arranged with respect to the longitudinal axis when in an initial condition thereof.

5. The portal system according to claim 4 wherein the portal seal is in the general shape of a frustum.

6. The portal system according to claim 5 wherein the portal seal defines an open cavity.

7. The portal system according to claim 5 wherein the portal seal defines a cavity therein, the cavity being open adjacent the portal sleeve.

8. The portal system according to claim 1 wherein the proximal seal wall is devoid of a seal passage.

9. The portal system according to claim 1 wherein the portal seal is mounted to the internal housing wall with a hinge, the portal seal being adapted for pivotal movement about the hinge.

10. The portal system according to claim 1 wherein the portal seal comprises a conformable material adapted to conform about the surgical instrument to establish a substantial sealed relation therewith.

11. The portal system according to claim 1 wherein the portal seal comprises a conformable material adapted to conform about the one or more sutures to establish a substantial sealed relation therewith and the internal housing wall.

12. The portal system according to claim 1 wherein including an instrument seal, the instrument seal having inner surfaces adapted to establish a substantial sealed relation with the surgical instrument.

13. The portal system according to claim 1 further including a zero closure valve, the zero closure valve adapted to substantially close the longitudinal opening the absence of the surgical instrument.

14. A method for performing a surgical procedure, comprising the steps of:
accessing an underlying surgical site with a portal, the portal including a portal housing and a portal sleeve extending from the portal housing, and defining a longitudinal opening therethrough, the portal housing including a portal seal having a proximal seal wall and an outer peripheral seal wall depending from the proximal seal wall;
performing a surgical task with at least one suture and directing a suture segment of the at least one suture from the surgical site and through the longitudinal opening of the portal; and
introducing a surgical instrument through the longitudinal opening whereby wall segments of the peripheral seal wall are displaced in at least a radial direction to contact the internal wall of the portal housing to engage one or more sutures extending through the longitudinal opening.

15. A method according to claim 14 wherein the step of introducing includes securing the one or more sutures against the internal housing wall with the wall segments upon radial displacement thereof.

16. The method according to claim 15 wherein the step of introducing includes establishing a sealed relation between the wall segments of the peripheral seal wall and the internal housing wall.

17. A method according to claim 14 wherein the seal comprises a conformable material and the proximal seal wall is devoid of a seal passage and wherein during the step of introducing the conformable material displaces to conform about the outer profile of the surgical instrument.
